(19) United States
(12) Patent Application Publication
(10) Pub. No.: US 2003/0154518 A1
(43) Pub. Date: Aug. 14, 2003

(54) Removal of selectable markers from transformed cells

(76) Inventors: Ethan R. Signer, San Francisco, CA (US); J. Ranjan Perera, San Francisco, CA (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 09/879,329

(22) Filed: Jun. 12, 2001

Related U.S. Application Data
(60) Provisional application No. 60/211,122, filed on Jun. 12, 2000.

(51) Int. Cl. A01H 1/00; C12N 15/82

(52) U.S. Cl. 800/288; 435/468; 435/320.1

(57) ABSTRACT

The subject application provides methods and materials useful for the transformation of eukaryotic cells, particularly plant cells. More specifically, the subject invention provides materials and methods for eliminating selectable marker genes from transformed cells. In a preferred embodiment the subject invention provides methods for removing both positive and negative selectable marker genes from plant cells after a gene of interest has been introduced into the plant cells. The methods of the subject invention are applicable to any gene of interest, in any plant species that can be transformed.

### CROSS-REFERENCE TO RELATED APPLICATIONS

This application claims priority to U.S. Serial No. 60/211,122 filed Jun. 12, 2000, that is incorporated herein by reference in its entirety.

The subject invention was made with government support under a research project supported by The National Science Foundation Grant No. 9206129-MCB. The government may have certain rights in this invention.

### BACKGROUND OF THE INVENTION

Advances in recombinant DNA technology coupled with advances in plant transformation and regeneration technology have made it possible to introduce new genetic material into plant cells, thus introducing new traits, e.g., phenotypes, that enhance the value of the plant or plant tissue. Recombinant DNA technology has been applied to other eukaryotic cells including, for example, mammals, insects, and fish.

Since the inception of recombinant DNA technology, there has been a demand for the improvement in the characteristics and productivity of the varieties, species, and strains used for production of foodstuffs. Genetically engineered plants resistant to pathogens or insects and the production of herbicide tolerant plants highlight the potential for crop improvement. The target crops can range from trees and shrubs to ornamental flowers and field crops. Indeed, it is clear that the “crop” can also be a culture of plant tissue grown in a bioreactor as a source for some natural product.

In general, a gene of interest is introduced into plants by linkage to an adjacent selectable marker present on the same transforming DNA. Selection methods are generally developed around a suitable assay and are of major importance in the discrimination between wild type and genetically manipulated organisms. Most currently available selectable markers for plant transformation are derived from microorganisms. This raises ecological and regulatory concerns. For example, there is concern that cross-pollination may lead to the introduction of resistance markers to weeds and non-transformed plants. Additionally, consumer groups have raised theoretical concerns regarding the transmission of resistance markers to bacteria colonizing the gut from consumed foodstuffs. Additionally, the use of multiple copies of a resistance gene within a cell can lead to undesirable gene silencing.

Therefore, there is a need for an effective means for removing from transformed cells marker genes used in the transformation process.

### BRIEF SUMMARY OF THE INVENTION

The subject application provides materials and methods useful for the transformation of eukaryotic cells, particularly plant cells. More specifically, the subject invention provides materials and methods for eliminating selectable markers from transformed cells. In a preferred embodiment, the subject invention provides methods for removing marker genes from plant cells after a gene of interest (GI) has been introduced into the plant cells employing marker genes. The methods of the subject invention are applicable to any gene of interest, in any plant species that can be transformed.

In a preferred embodiment, the method of the subject invention utilizes at least two types of selectable markers: a positive selectable marker (PS) which allows growth on selective medium of cells that carry the marker, but not of cells that do not carry the marker, and a negative selectable marker (NS) which prevents growth on selective medium of cells that carry the marker, but not of cells that do not carry the marker. In accordance with the practice of the subject invention, both the positive and the negative markers are determined to be eliminated by simple selection on appropriate selective media.

In a particular embodiment, a genetic construct of the subject invention comprises direct repeats of a gene of interest at the 5′ and 3′ ends of the construct which flank both a positive selectable marker gene and a negative selectable marker gene. Thus, the construct is arranged as follows: gene of interest (GI)-positive selectable marker gene (PS)-negative selectable marker gene (NS)-gene of interest (GI). This can be characterized by the formula:

GI-PS-NS-GI or GI-NS-PS-GI

wherein the order of PS and NS is not critical.

During both vegetative growth and meiosis, intrachromosomal homologous recombination between the direct repeats in the genetic construct promotes crossing-over that loops out and eliminates all of the intervening DNA, leaving behind within the construct only a single copy of the gene of interest itself. After segregation, the parental transgenic plants carry the negative selectable marker and are sensitive to the compounds which negatively impact the growth and/or survival of transformed plants or cells which contain the negative selectable marker gene; the loop-out recombination progeny will no longer carry the negative selectable marker gene and will be insensitive to the compounds which negatively impact the growth and/or survival of transformed plants or cells that contain the negative selectable marker gene.

In another embodiment of the present invention, additional genes (AG) can be added to the genetic constructs of the present invention resulting in the insertion of the Ags into the plant genome during the transformation process. The additional genes must flank the GI-PS-NS-GI portion of the genetic construct. Thus, this type of construct that contains one or more Ag in addition to the GI is arranged as follows:

AGx-GI-PS-NS-GI-AGy

wherein AG, GI, PS, and NS are as defined herein and x represents an integer of 1 or more and y represents an integer of 0 or more. If there is more than one AG, then it is preferred that that gene not be repeated, i.e., each AG be different.

The subject invention also provides genetic constructs, and compositions thereof, which provide at least one gene of interest and at least two selectable markers. The genetic constructs may comprise vectors, ssDNA, dsDNA, cDNA, tDNA, or mRNA. The construct may be linear or circularized depending upon the application.

### BRIEF DESCRIPTION OF THE DRAWINGS

FIGS. 1A and 1B depict plasmid and linearized nucleic acid constructs.

FIG. 2 illustrates a marker evicted Arabidopsis plant produced according to the subject invention. The file of this patent contains at least one drawing executed in color. Copies of this patent with color drawings will be provided by the Patent and Trademark Office upon request and payment of the necessary fee.

FIG. 3 illustrates that six out of seven T3 sub-lines (17-15, 17-38, 17-41, 17-42, 17-49, and 17-53), tested by PCR, have undergone marker eviction. Ef1-α primers were used for PCR amplification controls for template DNA. The absence of positive and the negative markers are clearly seen in the PCR results as compared to the non-marker evicted line 17-40. P=positive control, W=wild type, and N=no primers.

FIG. 4 illustrates that marker eviction is able to remove all selectable marker genes while the gene of interest remains within (i.e., is not evicted from) the transformed plant cells. The presence of the gene of interest has been confirmed by sequencing. FIG. 4A: Control, line 17-40 (non-marker evicted cells); 4B, line 17-15 (marker evicted cells); 4C, line 17-38 (marker evicted cells). Lane 1: 1 Kb ladder; lane 2: adhF/T-DNA; lane 3: T-DNA F/adhR; lane 4:NPT II; and lane 5: CodA.

### DETAILED DISCLOSURE OF THE INVENTION

The subject invention provides materials and methods useful for the transformation of eukaryotic cells. More specifically, the materials and methods of the subject invention are used to remove selectable marker genes from transformed cells. In a preferred embodiment of the subject invention, the materials and methods described herein are applied to the transformation of plant cells. Preferred plant cells include corn, soybean, cotton, wheat, canola, tobacco, Arabidopsis, rice, safflower and sunflower cells.

Specifically exemplified herein are methods which utilize at least two types of selectable marker genes; a positive selectable marker gene which allows growth on selective medium of cells that carry the marker, but not of cells that do not carry the marker gene, and a negative selectable marker gene which prevents growth on selective medium of cells that carry the marker gene, but not of cells that do not carry the marker gene. In accordance with the practice of the subject invention, both the positive and the negative marker genes are determined to be eliminated from the eukaryotic genome by simple selection for loss of the marker genes.

In a particular embodiment, the genetic construct comprises direct repeats of a gene of interest that flank positive and negative selectable marker genes (gene of interest-positive selection marker gene; negative selection marker gene-gene of interest). During both vegetative growth and meiosis, intrachromosomal homologous recombination between the direct repeats in the genetic construct promotes crossing-over that loops out and eliminates all the intervening DNA (in this case the positive and negative selectable marker genes), leaving behind within the construct only a single copy of the gene of interest itself. After segregation, the parental transgenic plants will carry the negative selectable marker and be sensitive to the compounds which negatively impact the growth and/or survival of transformed plants or cells which contain the negative selectable marker; the loop-out recombination progeny will no longer carry the negative selectable marker and will be insensitive to the compounds which negatively impact the growth and/or survival of transformed plants or cells.

In another embodiment, the genetic construct comprises direct repeats of a gene of interest (GI) which flank a positive selectable marker gene (PS) and a negative selectable marker gene (NS) and one or more additional genes (AG) that flank the GI. The AG may flank one side of the GI-PS-NS-GI portion of the construct or both sides. This can be represented as follows:

AG-GI-PS-NS-GI;

GI-PS-NS-GI-AG; or

AG-GI-PS-NS-GI-AG

wherein AG can represent one of more functional genes. It is also preferred that AG, when representing more than one gene, not be a repeat the same gene. During both vegetative growth and meiosis, intrachromosomal homologous recombination between the direct repeats in the genetic construct promotes crossing-over that loops out and eliminates all the intervening DNA (in this case the positive and negative selectable marker genes), leaving behind within the construct only a single copy of the gene of interest itself and any AGs that are present in the genetic construct. After segregation, the parental transgenic plants will carry the negative selectable marker gene and be sensitive to the compounds which negatively impact the growth and/or survival of transformed plants or cells which contain the negative selectable marker gene; the loop-out recombination progeny will no longer carry the negative selectable marker and will be insensitive to the compounds which negatively impact the growth and/or survival of transformed plants or cells. The gene construct left in the loop-out recombination progeny in this embodiment can be represented as follows:

AG-GI;

GI-AG; or

AG-GI-AG.

The subject application provides genetic constructs which provide at least one gene of interest and at least two selectable marker genes. The genetic constructs may comprise vectors, 228 ssDNA, dsDNA, cDNA, tDNA, or mRNA. The construct may be linear or circularized depending upon the application.

The genetic constructs of the subject invention may be introduced into cells by any of the well known DNA delivery methods, such as Agrobacterium mediated transformation employing RecA-mutants, electroporation, electrophoresis, microinjection, micro-projectile bombardment, micro-LASER beam-induced perforation of cell wall, polyethylene glycol mediated uptake, or simply by incubation.

The subject invention also provides compositions comprising genetic constructs that provide at least one gene of interest and at least two selectable marker genes and a carrier. Non-limiting examples of the carrier include microprojectiles coated with the genetic construct, and other solutions (such as solutions comprising polyethylene glycol (PEG) or other chemicals useful in the transformation of cells and known to the skilled artisan). Compositions comprising the subject genetic constructs can include appropriate nucleic acid vectors (plasmids), which are commercially available (e.g., Vical, San Diego, Calif.). In addition, the compositions can include a pharmaceutically acceptable carrier, e.g., saline. The pharmaceutically acceptable carriers are well known in the art and also are commercially available. For example, such acceptable carriers are described in E. W. Martin's Remington's Pharmaceutical Science, Mack Publishing Company, Easton, Pa.

The subject application also provides transgenic eukaryotic cells. In a preferred embodiment these cells are plant cells which are transformed with a gene of interest and any AG, but which do not have a selectable marker gene.

As used herein, the term “transgenic plants” or “transgenic plant cells” refers to plants (monocots or dicots) comprising plant cells in which heterologous polynucleotides are expressed as a result of manipulation by the hand of man. This includes plants which have been augmented by at least one incorporated DNA sequence (“gene of interest”). Preferred plants include corn, soybean, cotton, wheat, canola, tobacco, Arabidopsis, rice, safflower or sunflower.

A “positive selectable marker gene” or “PS” encodes a protein that allows growth on selective medium of cells that carry the marker gene, but not of cells that do not carry the marker gene. Selection is for cells that grow on the selective medium (showing acquisition of the marker) and is used to identify transformants. A common example is a drug-resistance marker such as NPT (neomycin phosphotransferase), whose gene product detoxifies kanamycin by phosphorylation and thus allows growth on media containing the drug.

Other positive selectable marker genes for use in connection with the present invention include, but are not limited to, a neo gene (Potrykus et al., 1985), which codes for kanamycin resistance and can be selected for using kanamycin, G418, etc.; a bar gene, which codes for bialaphos (basta) resistance; a mutant aroA gene, which encodes an altered EPSP synthase protein (Hinchee et al., 1988), thus conferring glyphosate resistance; a nitrilase gene such as bxn from Klebsiella ozaenae, which confers resistance to bromoxynil (Stalker et al., 1988); a mutant acetolactate synthase gene (ALS), which confers resistance to imidazolinone, sulfonylurea or other ALS inhibiting chemicals (European Patent Application 154,204, 1985); a methotrexate resistant DHFR gene (Thillet et al., 1988), or a dalapon dehalogenase gene that confers resistance to the herbicide dalapon; the pat gene from Streptomyces viridochromogenes, which encodes the enzyme phosphinothricin acetyl transferase (PAT) and inactivates the active ingredient in the herbicide bialaphos, phosphinothricin (PPT); or a mutated anthranilate synthase gene that confers resistance to 5-methyl tryptophan.

Where a mutant EPSP synthase gene is employed, additional benefit may be realized through the incorporation of a suitable chloroplast transit peptide, CTP (European Patent Application 0,218,571, 1987). The bialaphos resistance genes useful in the practice of the invention are obtainable from species of Streptomyces (e.g., ATCC No.21,705) and described in Murakami et al., 1986, and Thompson et al., 1987.

Additional positive selectable marker genes include those genes that provide resistance to environmental factors such as excess moisture, chilling, freezing, high temperature, salt, and oxidative stress. Of course, when it is desired to introduce such a trait into a plant as a “gene of interest”, the selectable marker cannot be one that provides for resistance to an environmental factor. Markers useful in the practice of the claimed invention include: an “antifreeze” protein such as that of the winter flounder (Cutler et al., 1989) or synthetic gene derivatives thereof; genes which provide improved chilling tolerance, such as that conferred through increased expression of glycerol-3-phosphate acetyltransferase in chloroplasts (Murata et al., 1992; Wolter et al., 1992); resistance to oxidative stress conferred by expression of superoxide dismutase (Gupta et al., 1993), and may be improved by glutathione reductase (Bowler et al., 1992); genes providing “drought resistance” and “drought tolerance”, such as genes encoding for mannitol dehydrogenase (Lee and Saier, 1982) and trehalose-6-phosphate synthase (Kaasen et al., 1992).

A “negative selectable marker gene” or “NS” encodes a protein that prevents the growth of a plant or plant cell on selective medium of plants that carry the marker gene, but not of plants that do not carry the marker gene. Selection of plants that grow on the medium provides for the identification of plants that have eliminated or evicted the selectable marker genes. An example is CodA (Escherichia coli cytosine deaminase), whose gene product deaminates 5-fluorocytosine (which is normally non-toxic as plants do not metabolize cytosine) to the toxic 5-fluorouracil.

Other negative selectable markers include the haloalkane dehalogenase (dhlA) gene of Xanthobacter autotrophicus GJ10 which encodes a dehalogenase, which hydrolyzes dihaloalkanes, such as 1,2-dichloroethane (DCE), to a halogenated alcohol and an inorganic halide (Naested et al., 1999, Plant J. 18(5):571-6).

“Positive selective medium” describes the medium or growth conditions which select for cells which contain a positive selectable marker gene. Transformed cells survive and/or grow when exposed to agents or conditions which would, normally, be detrimental to the survival of a plant or cell that did not contain the positive selectable marker gene. “Negative selective medium” describes medium or growth conditions which select for cells which do not contain a negative selectable marker gene. Transformed cells survive and/or grow when exposed to agents or conditions which would, normally, be detrimental to the survival of a plant or cell which contained the negative selectable marker gene.

A “gene of interest” or “GI”, and the “additional genes” or “AG”, include, but are not limited to, genes which are not normally present in the transformed plant. This includes DNA sequences not normally transcribed into RNA or translated into a protein (“expressed”), or any other genes or DNA sequences which one desires to introduce into the non-transformed plant, such as genes that may normally be present in the non-transformed plant, but which one desires to either genetically engineer or to alter the expression thereof.

It is contemplated that in some instances the genome of transgenic plants of the present invention will have been augmented through the stable introduction of the transgene. However, in other instances, the introduced gene will replace an endogenous sequence. A “gene of interest” or an “additional gene” may include nucleic acids encoding viral, parasitic, tumor, bacterial, or other known immunogens which may be expressed in plants (see, for example, U.S. Pat. No. 6,034,298, hereby incorporated by reference in its entirety); nucleic acids which confer resistance to drought, stress, herbicide, environmental factors such as frost, disease, or pests/insects; nucleic acids which reduce or eliminate mycotoxin; nucleic acids which improve grain composition or quality, crop yield or nutritive quality; alter morphology of plants or plant organs such as branching pattern, leaf or flower branching, size or shape, root branching, pattern or thickness; nucleic acids which confer frost resistance, improve nutrient utilization, cause male sterility (see, for example, U.S. Pat. No. 6,025,545, hereby incorporated by reference in its entirety).

Other embodiments envision the “gene of interest” and “additional gene” to include nucleic acid sequences encoding therapeutically or commercially relevant proteins, including, but not limited to, enzymes (proteases, recombinases, lipases, kinases, carbohydrases, isomerases, tautomerases, nucleases, etc.), hormones, erythropoietin, interleukins, cytokines, receptors, transcription factors, growth factors, globin proteins, immunosupppressive proteins, tumor proteins/immunogens, autoantigens, complement proteins, milk proteins, bovine and human serum albumin, immunoglobulins, pharmaceutical proteins and vaccines. The nucleic acid sequences encoding these proteins, immunogens, or phenotypic characteristics are known and reported throughout the scientific literature, sequence databases, and/or patent literature.

In one embodiment, the subject invention provides a method of transforming cells comprising:

1) providing a DNA construct which comprises direct repeats of a gene of interest at both ends flanking a positive selectable marker gene and a negative selectable marker gene;

2) transforming cells by introducing the construct into the cells;

3) growing or culturing the cell on positive selective medium;

4) selecting the transformed cells having the genetic construct which grows on the positive selective medium;

5) transferring the cells to a negative selective medium;

6) growing or culturing the cells on the negative selective medium; and

7) selecting those cells which grow on the negative selective medium.

The cells that grow on the negative selective medium are cells that have the PS and NS looped-out and contain only the GI.

The present invention provides a process to introduce a transgene in calli and excise the selected marker genes in one step, without having to introduce and subsequently remove, any recombinase gene, thus drastically reducing the time and effort in making commercially relevant transgenic plants. In this embodiment, a DNA construct, which is either a T-DNA or any other DNA, having the configuration that two copies of a gene of interest (GI) flank a positive selectable marker gene and a negative selectable marker gene, such as CodA, is first introduced into plant cells (plant tissue cells or callus) by agrobacterial infection, particle bombardment or any other means such that the construct DNA is integrated into the chromosome. If plant tissue cells are employed in the transformation process, then those cells are placed on callus induction medium to form callus. Transformed calli are then exposed to an agent that selects for expression of the positively selectable marker gene, i.e., positive selective medium. Subsequently, those calli that survive the positive selection are exposed to negative selective medium (5-FC in the case of CodA) to select for loss of the negative selectable marker (CodA) gene. In this process, calli cells that are hemizygous for a single copy T-DNA insertion get an opportunity for excising the internal region between the two copies of the repeated gene (GI). Cells with an excision event now are able to grow in the presence of the negative selectable marker agent (e.g., 5-FC). The occurrence of excision can be verified by PCR. Only a single copy of the GI should remain, both marker genes having been excised. Transgenic calli will then be allowed to produce a shoot in order to obtain a stable transgenic line free of any marker genes. This one-step marker excision has the additional advantage that this will resolve complex tandem multiple copies of transgenes in the plant genome, which are often produced during the transformation process, to a single copy of the transgene that is otherwise free of any marker genes.

In another embodiment, it is possible to first confirm a clone of callus cells that contains a single, hemizygous, insertion of the construct, and subsequently allow excision of marker genes by exposing to negative selective medium (e.g., 5-FC). In other embodiments of the present invention, primary transgenic calli with the inserted construct will be exposed to a homologous recombination promoting agent (for example, including, but not exclusively, the RecA protein from E. coli, or any other protein that catalyzes homologous recombination, or any gene that produces such a homologous recombination promoting enzyme which are either endogenous to the plant or are supplied as a foreign gene, either transiently or stably). This will enhance the frequency of excision of the marker genes [Reiss B, Schubert I, Kopchen K, Wendeler E, Schell J, Puchta H.; “RecA stimulates sister chromatid exchange and the fidelity of double-strand break repair, but not gene targeting, in plants transformed by Agrobacterium”; Proc Natl Acad Sci USA. (2000) 97:3358-63].

In another embodiment, the subject invention provides a method of transforming cells comprising:

1) providing a DNA construct which comprises (a) direct repeats of a gene of interest at both ends flanking a positive selectable marker gene and a negative selectable marker gene and (b) one or more additional genes that flank either side or both sides of (a);

2) transforming cells by introducing the construct into the cells;

3) growing or culturing the cells on positive selective medium;

4) selecting the transformed cells having the genetic construct which grows on the positive selective medium;

5) transferring the cells to a negative selective medium;

6) growing or culturing the cells on the negative selective medium; and

7) selecting those cells which grow on the negative selective medium.

The cells that grow on the negative selective medium are cells that have the PS and NS looped-out and contain only the GI and the AG.

In a preferred embodiment of the present invention, plant cells are transformed with a construct of the present invention and transgenic plants are regenerated using standard selection (positive selective medium) and regeneration techniques before subjecting the transgenic plant cells or transgenic plant tissues to the negative selective medium. The T0 plant (primary transformant) is allowed to set seed and the T1 seed is collected. The T1 seed is then germinated on a negative selective medium to identify those transformants that have looped-out the positive selectable marker gene and the negative selectable marker gene. In further preferred embodiments the T1 seed is germinated and grown to set seed and the T2 seed is collected. The T2 seed can then be subjected to negative selection or, alternatively, can ge grown to set T3 seed, and so on to produce further generation progeny (descendant plants). The negative selective medium can be employed to germinate any generation seed.

This preferred embodiment of removing selectable marker genes from transformed plant cells can be summarized as follows:

(a) transforming cells with a genetic construct to form T0 transformants,

(b) culturing the cells of (a) on a positive selective medium,

(c) selecting the T0 transformant cells that grow on the positive selective medium,

(d) regenerating a fertile T0 plant from the T0 transformant cells whereby T1 seed is formed,

(e) collecting the T1 seed from the T0 plant or the seed from a subsequent Tn generation plant wherein n is a whole number greater than one,

(f) germinating the T1 seeds or Tn seeds on a negative selective medium to form seedlings, and

(g) selecting the seedlings that grow on the negative selective medium wherein the selected seedlings contain the gene sequence of interest, but neither the positive selectable marker gene sequence nor the negative selectable marker gene sequence.

Following are examples which illustrate procedures for practicing the invention. These examples should not be construed as limiting. All percentages are by weight and all solvent mixture proportions are by volume unless otherwise noted.

### EXAMPLE 1

Removal of Marker Genes

In a specific embodiment, the method of the subject invention comprises the following steps:

1) providing a DNA construct which comprises direct repeats of a gene of interest which flank NPT (a positive selectable marker) and CodA (a negative selectable marker);

2) transforming a cell by introducing the construct into the cell;

3) growing or culturing the cell on positive selective medium which comprises kanamycin;

4) selecting a transformed cell having the genetic construct that grows on the positive selective medium;

5) transferring the cell to a negative selective medium having 5-fluorocytosine;

6) growing or culturing the cell on the negative selective medium; and

7) selecting those cells which grow on the negative selection medium.

Both the positive and the negative markers are eliminated by simple selection for loss of CodA on 5-fluorocytosine containing medium. Intrachromosomal homologous recombination (IHR) between the direct repeats in the genetic construct promotes crossing-over that loops out and eliminates intervening DNA and leaves a single copy of the gene of interest. Plants which have not undergone IHR still carry CodA and are sensitive to 5-fluorocytosine; the loop-out recombination progeny no longer carry CodA and, therefore, are insensitive to 5-fluorocytosine. The frequency of such intrachromosomal loop-out events, in Arabidopsis, for instance, is 10⁻⁵ among germinating seeds. That is within the resolving ability of 5-fluorocytosine selection.

### EXAMPLE 2

Screening for Transformed Plants

In a particularly preferred embodiment, the application provides a method of screening for transformed plants comprising:

1) providing a DNA construct which comprises direct repeats of the gene of interest which flank the NPTII gene (a positive selectable marker) and the CodA gene (a negative selectable marker);

2) transforming cells by introducing the construct into the cells;

3) growing or culturing the cells on positive selective medium which comprise kanamycin;

4) selecting transformed cells having the genetic construct which grow on the positive selective medium;

5) transferring the cells to a negative selective medium which comprises 5-fluorocytosine; and

6) growing or culturing the cells on said negative selective medium.

The cells that grow on the 5-fluorocytosine containing medium have undergone IHR and looped-out the selectable marker genes.

### EXAMPLE 3

Removal of Selective Markers from Transformed Plants Via Positive and Negative Selection

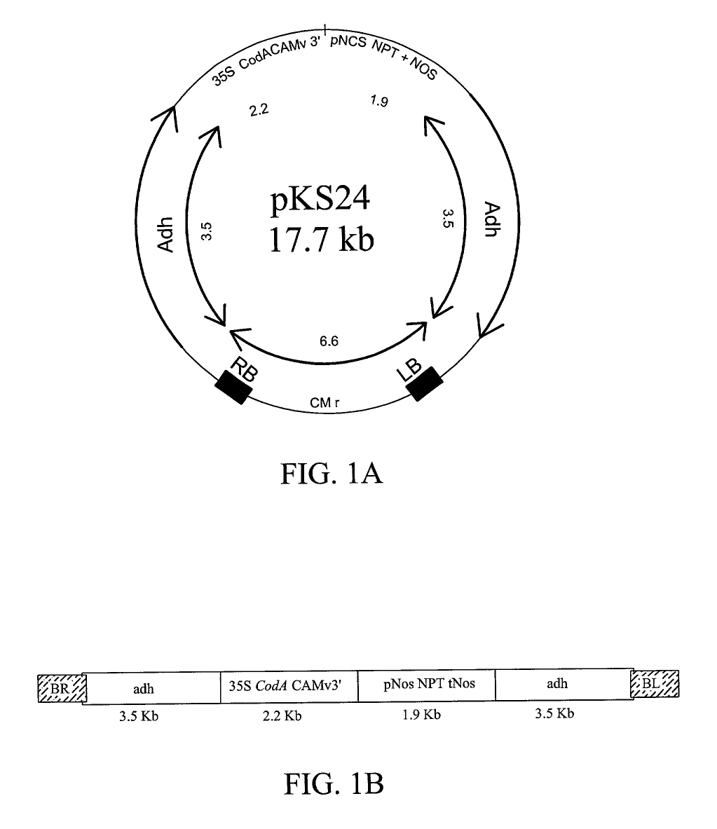

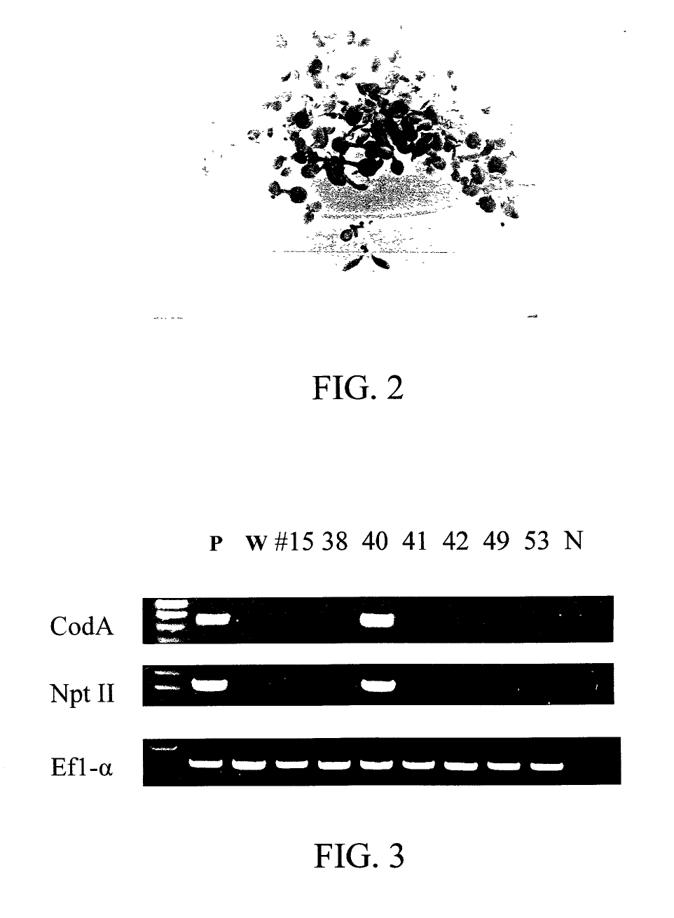

Construct pKS24 shown in FIG. 1A comprises direct repeats of a gene of interest (ADH gene) at the 5′ and 3′ ends of the construct, which flank both a positive (NPTII gene) and a negative (CodA gene) selectable marker gene. A single copy homozygous line was obtained and T3 seeds were screened for positive (NPTII gene) and negative (CodA gene) marker eviction. A transgenic seedling containing a single copy of the ADH gene and having both the NPTII gene and the CodA gene removed (evicted) can be seen in FIG. 2. This construct was transformed into Arabidopsis ecotype Columbia by Agrobacterium mediated root transformation to obtain single copy homozygous plants in later generations. Plant selection was done on kanamycin medium as the positive selective medium. Negative selective medium employed 5-fluorocytosine as the negative selective agent.

Construct pKS24 (map shown in FIG. 1A) contains two Arabidopsis ADH repeats (3.5 Kb) that flank the positive (NPTII) and negative (CodA) selectable marker genes. This 11.1 Kb fragment was cloned into a 6.6 Kb T-DNA binary vector pPZP100 having resistance for chlorampenicol for bacterial selection. The T-region was limited to 582 bp and contained multiple cloning sites located in-between right and left borders (BamH1, Xba1, Sal1, Pst1, HindIII and EcoR1 sites). The 3.5 Kb ADH fragment contained the coding region, as well as some of the promoter fragments of the ADH gene. 35S and NOS promoters were used to drive the positive selectable marker NPTII and the negative selectable marker CodA, respectively. Terminators were CAMv3′ and NOS 3,′ respectively. Size of the 35S::CodA::CAMv3′ was 2.2 Kb and the pNOS::NPT::tNOS was 1.9 Kb. The size of the final construct pKS24 was 17.7 Kb. This binary vector was not self-transmissible. Since this plasmid contains a bom site, it was able to mobilize in trans from E. coil into an Agrobacterium cell with helper plasmid pRK2013 by triparental mating. The resultant construct was then transformed into Arabidopsis ecotype Columbia via root transformation. Kn (25 mg/L) resistant plants were further screened to identify single copy transgenic lines. Genetic screening of line KS24-27 was done to identify a single copy homozygous line. Southern blot analysis was done to detect single copy homozygous plants. Several T0 kanamycin resistant transgenic plants were chosen to screen a single copy line. DNA from these plants was isolated and digested with several restriction enzymes (Sac1, Sal1, and Pac1). Sac1 digested plant DNA confirmed kanamycin (kn) resistant plants for the presence of the NPTII gene hybridized with 1.9 Kb P32 labeled kanamycin fragment. Sac1 DNA digestion produced a 1.9 Kb kanamycin fragment and, during the Southern hybridization, this band was identified by the kanamycin probe.

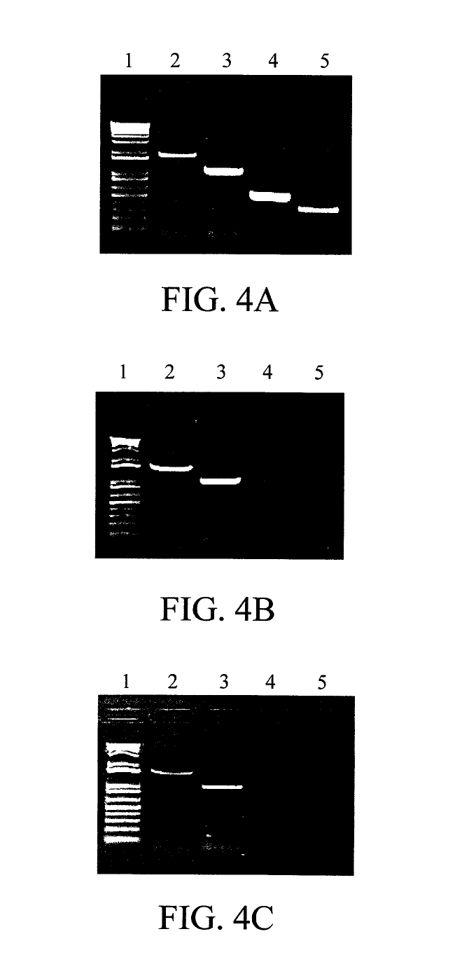

After T3 segregation, seeds from 14 single copy homozygous sub-lines of the parental line were screened for the possible marker eviction events. Several sub-lines showed different eviction frequencies. During both vegetative growth and meiosis, intra-chromosomal homologous recombination between the direct repeats (adh) in the genetic construct was promoted. The crossing over that looped out and eliminated all the intervening DNA resulted in a construct with only a single copy of the adh gene. Non-evicted plants, those that still carry the negative selectable marker, were sensitive to the 5-fluorocytosine negative selection agent. Marker evicted events were confirmed by PCR and also by sequencing the PCR products. Current data show that marker eviction based on positive/negative selection of the present invention is useful to engineer crops free of antibiotic/herbicide selectable markers (see FIGS. 4A-4C).

Identification of a Single Copy Homozygous Plant

Genomic DNA from several kn resistance T0 plants were digested with Sal1 and Pac1 restriction enzymes to identify single copy transgenic lines via Southern analysis. Neither Sal1 nor Pac1 cuts the alcohol dehydrogenase gene. A probe was made out of a 3.5 Kb adh Sac1 fragment. Based on Southern data, the lines designated K24-27 and K25-3 were chosen for further analysis. Segregation analysis of T1 seeds showed 3:1 Mendalian pattern. Seeds from single copy T1 plants were selected on 25 mg/L kanamycin to identify homozygous lines. The initial segregation was 156:64. The screen was continued until 14 single copy homozygous sub-lines in consecutive generations were identified. Results from the kanamycin resistant plants led to identifying the following sub-lines “1, 2, 3, 4, 6, 9, 10, 12, 13, 14, 17, 20, 21, 22” as homozygous. Fifty (50) independent plants from each sub-line were grown and seeds were collected for further sow in order to obtain sufficient seeds for negative selection. Seeds from the T3 generation were used for the marker eviction screening.

Negative Selection

A total of 105,000 seeds (7500×14)) from each sub-line were tested for 5FC resistance. As shown in Table 1 below, lines 12, 17,20,21 and 22 gave the highest marker eviction events. Sub-line 22 had a complete marker eviction. PCR data from sub-line 22 plants confirms that this effect was due to the removal of the CodA gene.

TABLE 1

| Line number (homozygous) | Number of seedlings resistant to 5FC' |
|---|---|
| 1 | 8 |
| 2 | 16 |
| 3 | 4 |
| 4 | 5 |
| 6 | 19 |
| 9 | 4 |
| 10 | 6 |
| 12 | 71 |
| 13 | 3 |
| 14 | 0 |
| 17 | 97 |
| 20 | 49 |
| 21 | 23 |
| 22 | 20 |

PCR Validation and Sequence Confirmation of Marker Evicted Events

Several primers were designed to verify the marker eviction events on lines 12, 17, 20, and 22. T-DNA border primers were from the pPZP100 sequence and adh primers were from the Arabidopsis adh gene coding sequence. Using the above primers, the remaining single copy adh gene was confirm by PCR, As shown in FIG. 4, PCR amplification with adh forward and T-DNA reverse primers, and also T-DNA forward and adh reverse produced genomic PCR bands. Marker eviction was confirmed by the absence of the Kn gene and the CodA gene.

It should be understood that the examples and embodiments described herein are for illustrative purposes only and that various modifications or changes in light thereof will be suggested to persons skilled in the art and to be included within the spirit and purview of this application and the scope of the appended claims.

### REFERENCES (ALL OF WHICH ARE INCORPORATED HEREIN BY REFERENCE)

Ditta G., Stanfield S., Corbin D., Helinski D. R: [1980] “Broad host range cloning system for gram negative bacteria: construction of a gene bank of Rhizobium meliloti,” Proc Natl Acad Sci USA 77:7347-7351

Ow, D. W. & Medberry, S. L. [1995] “Genome manipulation through site-specific recombination,” Crit.Rev.Plant Sci. 14, 239-261

Perera R J, Linard C G, Signer E R. [November 1993 ] “Cytosine deaminase as a negative selective marker for Arabidopsis,” Plant Mol Biol. 23(4):793-9

Sambrook, J., Fritsh, E. F., & Maniatis, T. [1989] Molecular Cloning. A Laboratory Manual (Cold Spring Harbor Laboratory Press, Cold Spring Harbor, N.Y.)

Valvekens, D., Van Montagu, M., and Van Lijsebettens, M. [1988] “Agrobacterium tumefaciens-mediated transformation of Arabidopsis root explants using kanamycin selection,” Proc. Natl. Acad. Sci. USA. 85:5536-5540

1. A genetic construct which comprises:
a. a positive selectable marker gene,
b. a negative selectable marker gene, and
c. direct repeats of a gene of interest that flank the positive and negative selectable marker genes of (a) and (b).

2. The genetic construct of claim 1 wherein the negative selectable marker gene is CodA.

3. The genetic construct of claim 2 wherein the positive selectable marker gene is NPTII, BAR, PAT or EPSP synthase

4. A method of removing selectable marker genes from transformed eukaryotic cells which comprises:
a. transforming cells with a genetic construct of claim 1,
b. culturing the cells of (a) on a positive selective medium,
c. transferring the transformed cells in (b) onto a negative selective medium, and
d. selecting the cells that grow on the negative selective medium
wherein the selected cells that grow on the negative selective medium contain the gene sequence of interest but neither the positive selectable marker sequence nor the negative selectable marker sequence.

5. The method of claim 4 wherein the negative selectable marker gene is CodA.

6. The genetic construct of claim 1, wherein said construct comprises a polynucleotide sequence in the 5′ to 3′ (right to left) direction:
a. a gene sequence of interest,
b. a positive selectable marker sequence,
c. a negative selectable marker sequence and
d. a repeat of the gene sequence of interest in (a) above.

7. The genetic construct of claim 6 wherein the negative selectable marker sequence is CodA.

8. A method of removing selectable marker genes from transformed plant cells which comprises:
a. transforming cells with a genetic construct of claim 1 to form T0 transformants,
b. culturing the cells of (a) on a positive selective medium,
c. selecting the T0 transformant cells that grow on the positive selective medium,
d. regenerating a fertile T0 plant from the T0 transformant cells whereby T1 seed is formed,
e. collecting the T1 seed from the T0 plant or the seed from a subsequent Tn generation plant wherein n is a whole number greater than one,
f. germinating the T1 seeds or Tn seeds on a negative selective medium to form seedlings, and
g. selecting the seedlings that grow on the negative selective medium
wherein the selected seedlings contain the gene sequence of interest but neither the positive selectable marker sequence nor the negative selectable marker sequence.

9. The method of claim 8 wherein the negative selectable marker gene is CodA and the negative selective medium comprises 5-fluorocytosine.

10. The genetic construct of claim 1, wherein said construct comprises, in the 5′ to 3′ direction (left to right), the formula:
GI-PS-NS-GI
wherein GI represents a gene of interest, PS represents a positive selectable marker gene and NS represents a negative selectable marker gene.

11. The genetic construct of claim 10 wherein NS is CodA.

12. The genetic construct of claim 1, wherein said construct comprises, in the 5′ to 3′ direction (left to right), the formula:
GI-NS-PS-GI
wherein GI represents a gene of interest, NS represents a negative selectable marker gene, and PS represents a positive selectable marker gene.

13. The genetic construct of claim 12 wherein NS is CodA.

14. The genetic construct of claim 1, wherein said construct comprises, in the 5′ to 3′ direction (left to right), the formula:
AGx-GI-PS-NS-GI-AG′y
wherein AG and AG′ represent additional genes of interest, x represents an integer of 1 or larger, y represents an integer of 0 or larger, GI represents a gene of interest, NS represents a negative selectable marker gene, and PS represents a positive selectable marker gene.

15. The genetic construct of claim 14 wherein the genes represented by AG and AG′ are never the same.

16. The genetic construct of claim 14 wherein the NS is CodA.

17. The method of claim 4 wherein the eukaryotic cell is a plant cell.

18. The method of claim 4 wherein the plant cell a corn, soybean, cotton, wheat, canola, tobacco, Arabidopsis, rice, safflower or sunflower cell.

19. The method of claim 8 wherein the plant cell is a monocot or dicot cell.

20. The method of claim 19 wherein the plant cell a corn, soybean, cotton, wheat, canola, tobacco, Arabidopsis, rice, safflower or sunflower cell.
